(19) United States
(12) Patent Application Publication
(10) Pub. No.: US 2024/0299705 A1
(43) Pub. Date: Sep. 12, 2024

(54) APPARATUS AND METHOD FOR EVERTING CATHETER WITH ALIGNMENT AND COMPLIANT PRESSURIZATION

(71) Applicant: CrossBay Medical, Inc., San Diego, CA (US)

(72) Inventors: Steven R. BACICH, Half Moon Bay, CA (US); Matthew Thomas YUREK, San Diego, CA (US); Cristiano Danilo Maria FONTANA, Milan (IT); Piush VIDYARTHI, San Rafael, CA (US)

(73) Assignee: CrossBay Medical, Inc., San Diego, CA (US)

(21) Appl. No.: 18/604,306

(22) Filed: Mar. 13, 2024

Related U.S. Application Data
(63) Continuation of application No. 16/029,305, filed on Jul. 6, 2018, now abandoned.
Application No. 16/029,305 is a continuation of application No. PCT/US2018/040820, filed on Jul. 3, 2018.
Application No. 16/029,305 is a continuation-in-part of application No. 14/525,043, filed on Oct. 27, 2014, now Pat. No. 10,245,074.
(60) Provisional application No. 62/528,422, filed on Jul. 3, 2017.
(60) Provisional application No. 61/902,742, filed on Nov. 11, 2013.
(60) Provisional application No. 62/007,339, filed on Jun. 3, 2014.
(60) Provisional application No. 61/977,478, filed on Apr. 9, 2014.
(60) Provisional application No. 62/005,355, filed on May 30, 2014.

(51) Int. Cl. A61M 25/01; A61B 1/00; A61M 25/00; A61M 25/10

(52) U.S. Cl. CPC A61M 25/0119; A61M 25/0122; A61M 25/1006; A61B 1/00151; A61M 2025/0018; A61M 2025/0062; A61M 2025/1068

(57) ABSTRACT

Everting balloon systems and methods for using the same with an alignment element for stability and anti-rotation of the everting balloon are disclosed herein. The systems can be configured to access and deliver instruments, media, or other catheters into bodily lumens and cavities. The alignment element eliminates the potential for the everting membrane to become twisted or rotated which could impact access or the ability of the system to deliver materials. A compliance member facilitates internal pressurization of the everting catheter system.

### CROSS-REFERENCE TO RELATED APPLICATIONS

This application is a continuation of U.S. application Ser. No. 16/029,305, filed Jul. 6, 2018, which is a continuation of International Application No. PCT/US18/40820, filed Jul. 3, 2018, which claims priority to U.S. Provisional Application No. 62/528,422, filed Jul. 3, 2017, all of which are incorporated by reference herein in their entireties.

### BACKGROUND

This application has particular utility for everting catheters that are characterized with an inner catheter, outer catheter, and everting membrane that is connected to both catheters. The inner catheter may contain an inner lumen to pass fluid or media, drugs or therapeutic agents, instruments or devices, and other catheters.

For physicians and medical professionals, accessing systems for vessels and bodily cavities in patients have typically used various guidewire and catheter technologies or everting catheters. Everting catheters utilize a traversing action in which a balloon is inverted and with the influence of hydraulic pressure created by a compressible or incompressible fluid or media, rolls inside out or everts with a propulsion force through the vessel. Everting balloons have been referred to as rolling or outrolling balloons, evaginating membranes, toposcopic catheters, or linear everting catheters such as those in U.S. Pat. Nos. 5,364,345; 5,372,247; 5,458,573; 5,472,419; 5,630,797; 5,902,286; 5,993,427; 6,039,721; 3,421,509; and 3,911,927; all of which are incorporated herein by reference in their entireties. These are categorized as everting balloons and are for traversing vessels, cavities, tubes, or ducts in a frictionless manner. In other words, an everting balloon can traverse a tube without imparting any shear forces on the wall being traversed. Because of this action and lack of shear forces, resultant trauma can be reduced and the risk of perforation reduced. In addition as a result of the mechanism of travel through a vessel, material and substances in the proximal portion of the tube or vessel are not pushed or advanced forward to a more distal portion of the tube or vessel.

In addition, as the everting catheter deploys inside out, uncontaminated or untouched balloon material is placed inside the vessel wall. In the inverted or undeployed state, the balloon is housed inside the catheter body and cannot come into contact with the patient or physician. As the balloon is pressurized and everted, the balloon material rolls inside out without contacting any element outside of the vessel. Another advantage of an everting balloon catheter is that the method of access is more comfortable for the patient since the hydraulic forces “pull” the balloon membrane through the vessel or duct as opposed to a standard catheter that needs to be “pushed” into and through the vessel or duct.

Everting catheters have been described as dilatation catheters. Representative examples of dilating everting catheters include U.S. Pat. Nos. 5,364,345 and 4,863,440, both of which are incorporated by reference herein in their entireties.

Everting catheters have also been described with additional elements such as a handle for controlling instruments within an everting catheter. A representative example is U.S. Pat. No. 5,346,498 which is incorporated by reference herein in its entirety. Everting balloon catheters can be constructed with an inner catheter with an internal lumen or through-lumen (or thru-lumen). The through-lumen can be used for the passage of instruments, media, materials, therapeutic agents, endoscope, guidewires, or other instruments. Representative samples of everting catheters with through-lumens are in U.S. Pat. No. 5,374,247 and 5,458,573. In addition, everting catheters have been described with waists or a narrowing of the balloon diameter, such as in U.S. Pat. No. 5,074,845, which is incorporated by reference herein in its entirety.

Furthermore, infertility is a condition that affects 1 out of 8 couples in the US. One of the early treatments in the infertility regime is insemination. Intrauterine insemination or IUI is a very common procedure since it is in the early work up of an infertile couple. Most assisted reproductive clinics perform at least 3 IUI cycles before trying more expensive treatment options such as IVF.

Also, when delivering the reproductive material, such as an embryo, into the uterine cavity, vacuum effect can unintentionally remove the reproductive material from the uterine cavity. In existing systems, when the transfer catheter is retracted from a second outer or guiding catheter (e.g., the “inner” catheter), the retraction produces vacuum pressure within the uterine cavity. This vacuum pressure is created in the uterine cavity by the removal and backward movement of the transfer catheter within the inner catheter. After the embryo transfer is completed, an embryologist may inspect the transfer catheter to verify that the embryos or reproductive material was indeed deposited in the uterus and not pulled back into the transfer catheter because of the vacuum effect. The same procedure may be done for the outer catheter once this catheter is removed.

The passage of the embryo transfer catheter may become impeded if the everting membrane is rotated or twisted. Twists within the balloon membrane can also reduce the ability of the everting membrane to traverse a lumen or cavity or unroll as intended. A twist in the balloon membrane can occur if the inner catheter is rotated about its central axis in relation to a stationary outer catheter. By rotating the inner catheter, the balloon membrane which is connected between both the outer catheter and inner catheter becomes twisted. In this particular situation of an everting balloon, twists in the balloon membrane can significantly impact performance of the everting system.

A twist in the everting membrane can occur during use or prep of the catheter prior to inserting the device within a patient. A twist in the everting membrane can also occur when a catheter system has the requirement of multiple eversions and retractions to complete a procedure within a patient. Likewise, a twist in the balloon system can unintentionally occur as a byproduct of the manufacturing process.

In the device configuration using a handle system, an anti-rotation feature can be particularly advantageous. As described previously, handles are very useful for driving the inner catheter and controlling the advancement and retraction of instruments, other catheters, media, and materials within the inner catheter lumen. Manipulation of a handle can inadvertently rotate the inner catheter system within the outer catheter and thereby creates twists in the balloon membrane. This situation can be exasperated by the introduction and removal of multiple instruments and devices within the inner catheter lumen.

Having an everting catheter system in which twists or inadvertent rotations of the balloon membrane will enable more stable and secure use of an everting catheter. An untwisted balloon membrane provides the least obstructed passage within the everting system. Some everting catheter systems will be more prone to balloon twisting due to the length of the balloon membrane and inner catheter and type of balloon membrane material. In some clinical applications, more tortuous anatomy may instigate a greater likelihood of balloon twists as a result of the manipulations the clinician may need to perform to complete the procedure or obtain access to the desired target location in the body.

Maintaining the alignment of the inner catheter, outer catheter, and balloon membrane may be accomplished through a handle and ratchet system as described previously. The alignment feature is accomplished by the ratchet and handle that prevents rotation of the inner catheter. The systems described herein are directed towards internal catheter apparatus that provide alignment or anti-rotation capability without requiring an additional set of components like rails, tracks, ratchets, or handles on the exterior for the catheter system.

Another clinical issue with an everting catheter is that physicians may inadvertently pull or elongate the inner catheter upon inversion of the balloon membrane. Over-elongation can stretch the balloon membrane or damage the catheter components. A feature that mechanically prevents this from occurring will be a benefit to the catheter system.

Another problematic issue for everting catheters is the pressurization step in prepping the catheter. One option that is described in the prior art is the use of an inflation device with pressure gauge that indicates the internal pressure of the catheter system. Inflation devices with pressure gauges, or building an integral pressure gauge within the catheter system, can be expensive. Using a separate, reusable pressure gauge adds to the number of components required for performing the procedure. Having a simple mechanism that regulates and indicates the amount of pressure within the catheter system would be a benefit. For more specialized procedures, being able to modulate the internal pressure depending upon the medical procedure could be particularly advantageous.

For everting catheters used in IVF procedures, it is beneficial to stabilize the inner catheter when full eversion is completed for two-stage embryo transfer procedures. A two-stage embryo transfer is performed by everting the membrane across the endocervical canal and into the uterine cavity and subsequently placing the loaded embryo transfer catheter through the inner catheter and ultimately within the uterus. This operation is done in two steps and the infertility specialist will inform the embryologist that the inner catheter has been everted and is now in place within the uterine cavity. The embryologist will then aspirate and load the embryo or embryos into the distal end of the embryo transfer catheter for eventual insertion through the inner catheter for deposition in to the uterine cavity. This is the completion of the second stage of the process. During the loading step performed by the embryologist, a mechanism that stabilizes and indicates to the user that the inner catheter is in position would be a benefit.

Another problem with everting catheters is preparing the system by internal pressurization. This preparation step can vary among users and over-pressurization, and under-pressurization, of the everting system can negatively impact the performance of the device.

Another improvement to embryo transfer procedures would be systems that facilitate the use of transvaginal ultrasound. Systems that also remove the requirement for a speculum would be a benefit for patient comfort.

Another area of improvement is accessories that make handling the embryo transfer catheter easier for the embryologist and physician performing the transfer procedure.

### SUMMARY

An everting balloon system is disclosed that can be used for uterine access procedures. The everting balloon system can be used for IVF and intrauterine insemination procedures, urinary incontinence diagnostic and therapeutic procedures, delivering intra-fallopian tube inserts, media, or diagnostic instruments, dilation of a body lumen, for access and sealing within a body cavity, or combinations thereof. The system can have a handle for insertion.

The everting balloon system can be used to access the uterus, bladder, ureters, kidneys, ducts, vessels of the vasculature, nasal passageways, other bodily lumens, or combinations thereof. Devices, tools, instrumentation, endoscopes, drugs, therapeutic agents, sampling devices (brushes, biopsy, and aspiration mechanisms), or combinations thereof can be delivered through the inner catheter lumen to the target site.

The everting balloon system can have an internal alignment mechanism that prevents rotation and spinning of the balloon membrane.

The everting balloon system can have an internal mechanism that prevents over-elongation of the inner catheter during balloon inversion.

The everting balloon system can have a compliant pressurization apparatus that's provides a pre-determined pressure within the catheter system with an indicator to the user that system is at the appropriate operating pressure.

Another embodiment can automatically pressurize the everting balloon system to a predetermined amount.

The everting balloon system can have an integral pressurization system that provides an indicator and the ability to quickly shift the pressurization state of the balloon system from pressurized to non-pressurized. Intermediate degrees of pressurization can also be selected.

The everting balloon system can have a mechanism that stabilizes the inner catheter at the full eversion stage and provides an indicator to the user that catheter system is at the appropriate step in the process for embryo transfer.

The everting balloon system can have a proximal hub connector that aids the physician and embryologist in delivering the embryo transfer catheter to the delivery catheter.

The everting balloon system can be shaped with distal end features that facilitate uterine access without the need for a speculum and/or tenaculum.

The everting catheter system can have accessories that make the handling of the embryo transfer catheter easier.

### BRIEF DESCRIPTION OF THE DRAWINGS

FIGS. 1A through 1E are longitudinal cross-sectional views of the distal end of a variation of a method for using the everting balloon system.

FIG. 2A illustrates an everting balloon system with a delivery catheter, embryo transfer catheter, and a pressurization syringe in a disassembled configuration.

FIG. 2B illustrates a variation of the everting balloon system in an assembled and fully everted configuration.

FIG. 2C illustrates the everting balloon system of FIG. 2B with the embryo transfer catheter beyond the distal end of the everting balloon membrane.

FIG. 3A illustrates cross-sectional view of a variation of a method for using the everting balloon system with a flexible tip guidance wire beyond the distal end of the everting balloon membrane during the eversion process directing the everting balloon system beyond a cul-de-sac in the endocervical canal.

FIG. 3B illustrates a cross-sectional view of the method shown in FIG. 3A with the flexible tip guidance wire beyond the distal end of the everting balloon membrane at the completion of the eversion process beyond a cul-de-sac in the endocervical canal.

FIG. 4 illustrates a variation of the everting balloon system with a stopcock configuration for maintaining pressurization.

FIG. 5A illustrates is a close-up view of a variation of the everting balloon system with an internal alignment mechanism that prevents rotation and spinning of the balloon membrane.

FIG. 5B is a cross-sectional axial view of a variation of the internal alignment mechanism and mating geometry of the delivery catheter tubing.

FIG. 5C illustrates a variation of the alignment piece.

FIG. 6 illustrates a variation of an everting balloon system with an internal mechanism that prevents over-elongation of the inner catheter during balloon inversion.

FIGS. 7A, 7B and 7C illustrate everting balloon systems with a compliant pressurization apparatus which provides a pre-determined pressure within the catheter system with an indicator to the user that system is at the appropriate operating pressure.

FIG. 8 illustrates a variation of the everting balloon system with a mechanism that automatically pressurizes the everting balloon system to a predetermined amount.

FIG. 9A illustrates a variation of the everting balloon system with an integral pressurization system that provides an indicator and the ability to quickly shift the pressurization state of the balloon system from pressurized to non-pressurized at the fully everted state of the everting balloon system.

FIG. 9B illustrates a variation of the everting balloon system with an integral pressurization system that provides an indicator and the ability to quickly shift the pressurization state of the balloon system from high pressurization to low pressurization, and back to high pressurization, or multiple intermediate states of pressurization, during the eversion process.

FIG. 10 illustrates a variation of the everting balloon system with a mechanism that stabilizes the inner catheter at the full eversion stage and provides an indicator to the user that catheter system is at the appropriate step in the process for embryo transfer.

FIG. 11 illustrates an everting balloon system with a proximal hub connector that aids the physician and embryologist in delivering the embryo transfer catheter to the delivery catheter.

FIGS. 12A and 12A′ illustrates a variation of an everting balloon system shaped with distal end features that facilitate uterine access without the need for a speculum.

FIG. 12B illustrates in an axial view of the distal end (e.g., an acorn tip) features that facilitate uterine access without the need for a speculum.

FIG. 13 illustrates in a side view of an everting balloon system with a handle that controls the translation of the inner catheter.

FIGS. 14A and 14A′ illustrate a variation of the everting catheter balloon system with a translatable and adjustable distal end tip that can alter the working length of the everting balloon.

FIGS. 14B and 14B′ illustrate the translatable and adjustable distal end tip at an extended position with resultant working length of the everting balloon.

FIGS. 15A and 15A′ illustrate a protective tube system for the embryo transfer catheter that facilitates handling and transport of the catheter.

FIG. 15B, 15B′, and 15B″ illustrate a protective tube system for the embryo transfer catheter in the detached configuration for the loading of embryos.

FIG. 15C illustrates a protective tube system for the embryo transfer catheter in the re-attached mode for the transport of the embryo transfer catheter.

### DETAILED DESCRIPTION

An everting balloon system (also referred to as an everting catheter system) that can be used to traverse a vessel, such as the cervical canal is disclosed. The everting balloon system can be used to access the uterine cavity via the cervix. The cervical canal is a single lumen vessel that can stretch or dilate. The everting balloon system can have a control system that can be operated with one hand. The pressurization states of the everting catheter system can be changed and controlled with one hand of the user.

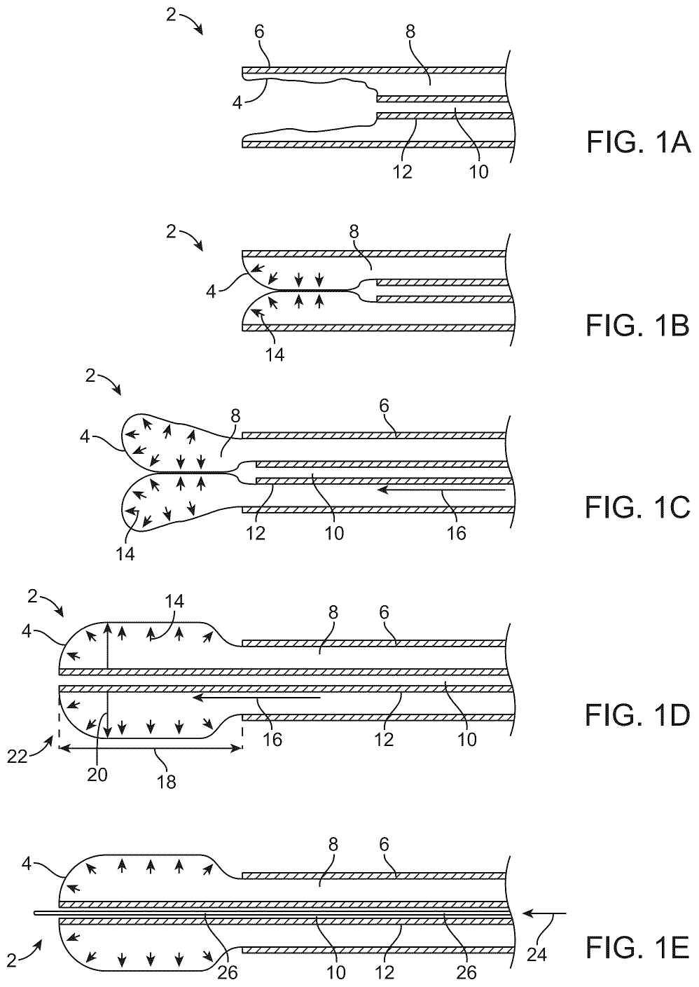

FIGS. 1A through 1E illustrate that an everting catheter system can have a radially outer catheter, a balloon membrane, and a radially inner catheter. The inner catheter can have an inner catheter lumen (e.g., a through-lumen). The distal end of the inner catheter lumen can be open or closed. The inner catheter can have the inner catheter lumen, or be a solid rod or flexible mandrel, or contain multiple lumens for the delivery of other agents, tools, catheters, instruments, endoscopes, and other media. The inner catheter can be made from multiple polymeric materials and have a more flexible distal end and more rigid proximal end. Distal end flexibility can be enhanced with the incorporation of a distal end coil or spring to provide distal end flexibility and support from kinking the lumen of the inner catheter. The internal lumen of the inner catheter can be made from a lubricious material such as Teflon or coated with a lubricious coating to facilitate the passage of instruments, tools, or other catheters through the internal lumen.

The everting balloon system can have a media volume. The media volume can be the contiguous open volume between the inner catheter and outer catheter that is proximal to the balloon membrane. A radially outer terminal perimeter of the balloon membrane can be attached to the distal terminal end of the outer catheter. A radially inner terminal perimeter of the balloon membrane can be attached to the distal terminal end of the inner catheter.

FIG. 1A illustrates that the everting catheter system can be in an unpressurized configuration. The media volume can be uninflated and unpressurized. The balloon membrane can be slack.

FIG. 1B illustrates that that everting catheter system can be in a pressurized and uneverted configuration. A pressurization device, such as a pump, for example at the proximal end of the everting catheter system can be in fluid communication with the media volume. The pressurization device can deliver a fluid media, such as a pneumatic gas or hydraulic liquid media (e.g., saline, water, culture media, air, carbon dioxide, air-infused fluids, carbonated fluids, or combinations thereof), at a media pressure to the media volume. The media pressure in the everting balloon can be from about 2 to about 5 atmospheres of pressure when in the everted configuration and higher media pressures from about 5 atmospheres to 10 atmospheres are possible, for example, to provide greater everting capability for more difficult or stenotic passageways in the body.

The balloon membrane can inflate and be in tension. The balloon membrane can block the distal port of the inner catheter lumen.

FIG. 1C illustrates that the everting catheter system can be in an inflated and partially everted configuration. The inner catheter can be translated distally, as shown by arrow, with respect to the outer catheter, and out of the outer catheter. The distal terminal end of the inner catheter can be proximal of the distal terminal end of the balloon membrane. The distal terminal end of the inner catheter can be proximal or terminal of the distal terminal end of the outer catheter. The balloon membrane can block the distal port of the inner catheter lumen or can be open allowing fluid communication between the inner catheter lumen and the target site.

FIG. 1D illustrates that the everting catheter system can be in an inflated, fully everted, and fully distally extended configuration. The inner catheter can be translated distally, as shown by arrow, with respect to the outer catheter until the distal terminal end of the inner catheter is longitudinally beyond or co-terminal with the distal terminal end of the balloon membrane. The distal port of the inner catheter lumen can be unobstructedly accessible and in fluid communication with the target site.

In the fully inflated configuration, the balloon membrane can form an inflated everting balloon. The everting balloon can have a balloon outer diameter and balloon length in the inflated and fully everted configuration.

The balloon outer diameter can be from about 2 mm to about 20 mm, more narrowly from about 2 mm to about 7 mm, for example about 3.0 mm. The outer diameter can be constant or vary along the length of the everting balloon. For example, for use in the cervical canal, the most proximal portion of the everting balloon outer diameter could be configured with a smaller outer diameter than the remainder of the everting balloon membrane. As an example, the first proximal portion of the everting balloon can have a smaller balloon outer diameter such as from about 2 mm to 4 mm for a length of from about 5 mm to about 10 mm from the distal terminal end of the outer catheter, and the remainder of the length (e.g., from about 4 cm to about 7 cm along the everting balloon) of the everting balloon can have a balloon outer diameter from about 4 mm 21 to about 7 mm.

The interior surface and lumen of the balloon can be coated with a lubricious material to facilitate rolling and unrolling of the interior surfaces of the everting balloon membrane.

The exterior surface of the balloon membrane can be configured with ridges, projections, bumps, grooves, and additional surface or mechanical features, or combinations thereof, for example for increased friction or holding power within the vessel.

The everting balloon length can be from about 2 cm to about 10 cm, more narrowly from about 3.5 cm to about 8.5 cm (e.g., for use in a longer uterine cavity lengths), yet more narrowly from about 5 cm to about 7.5 cm.

FIG. 1E illustrates that the everting catheter system can be in an inflated and partially or fully everted configuration. A tool, liquid, gas, or combinations thereof can be translated, as shown by the arrow, through the inner catheter lumen, out of the distal port of the inner catheter lumen and into the target site. The tool can be a biopsy tool, a scope, a sonogram probe, a plug, a cauterization tool, or combinations thereof. Suction can be applied from the proximal end of the inner catheter lumen, and to the target site, for example removing debris from the target site through the inner catheter lumen. For use in IVF procedures, an embryo transfer catheter is translated through the inner catheter lumen for deposition of embryo(s) or other reproductive material such as gametes or sperm.

To retract and reposition or remove the balloon membrane, the inner catheter can be pulled proximally to pull the balloon membrane back within the outer catheter. The balloon membrane can be deflated or have media pressure reduced and the entire system can be withdrawn from the target site.

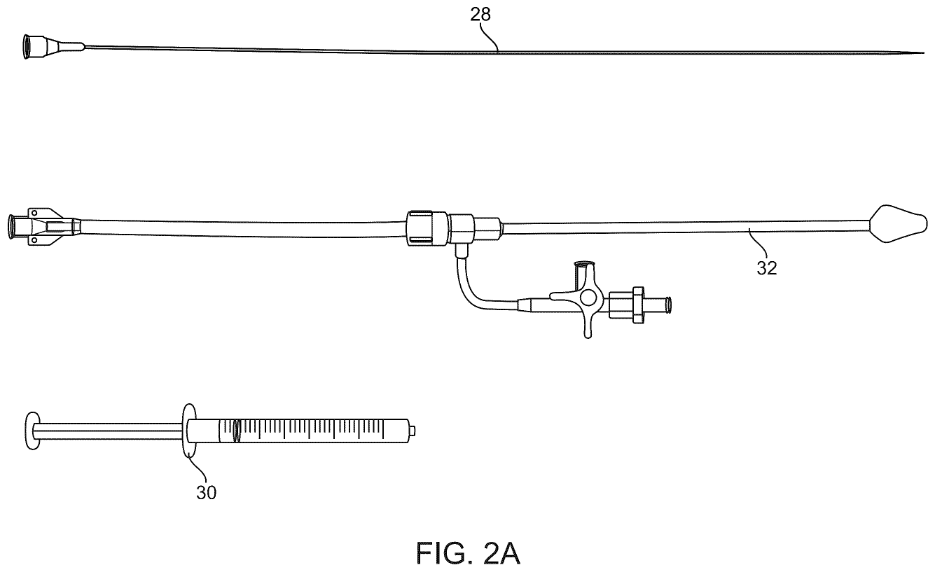

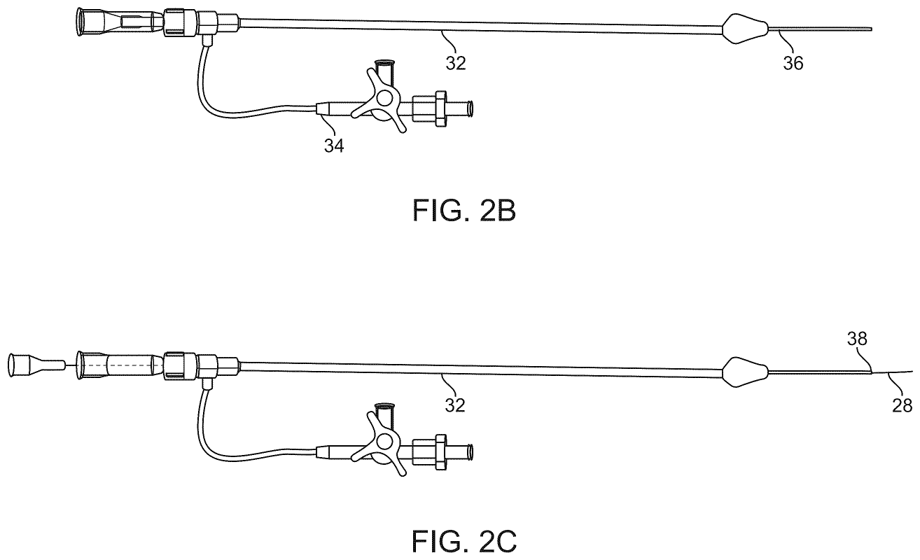

FIG. 2A illustrates an everting balloon system with a delivery catheter, embryo transfer catheter, and a pressurization syringe.

FIG. 2B illustrates a variation of the everting balloon system in a fully everted configuration. The everting balloon system can be equipped with a distal end opening or a pre-determined valve.

FIG. 2C illustrates an embryo transfer catheter distally beyond the distal end of the everting balloon membrane. The everting catheter system can access a bodily cavity (e.g., the uterine cavity or fallopian tubes) to deliver or introduce tools (e.g., instruments), reproductive media or material (e.g., embryos, in vitro fertilization (IVF) or insemination products, such as hormones), contrast media, dye, therapeutic agents, sclerosing agents to treat the endometrium, insufflation media, or combinations thereof to the cavity. For example, reproductive media can be delivered with a transfer catheter inserted through the inner catheter lumen to the uterine cavity.

FIG. 2B illustrates that a transfer catheter or insemination catheter can have a transfer connector, such as a female luer connector, a strain relief length, and a transfer tube. The transfer tube can hold the reproductive media. A delivery force, for example a positive fluid pressure, can be delivered through the transfer connector and strain relief length to push the contents of the transfer tube into the target site.

The transfer catheter can attach to or inserted through the inlet port. The transfer tube can hold an embryo, for example for in vitro fertilization or IVF. The embryo transfer catheter can deliver embryos through the system and to the uterine cavity and other agents that help facilitate embryo implantation such as materials that promote adherence of the embryo to the uterine endometrium. The embryo transfer catheter can have a distal end configuration that can promote implantation of the embryo(s) within the endometrial wall or within the sub-endometrial surface.

The embryo transfer catheter can hold spermatozoa and deliver the spermatozoa through the system and to the uterine cavity for intrauterine insemination procedures. The transfer catheter can hold and deliver or deposit materials, such as drugs, therapeutic agents, instruments, endoscopes, cytology brushes, other catheters, or combinations thereof through the system and into the uterine cavity. The transfer catheter can be connected to a vacuum source for the aspiration of materials from the uterine cavity or other bodily cavities and lumens.

The transfer catheter and/or materials can be loaded in the inner catheter lumen prior to everting the everting balloon within the vessel or bodily cavity. For example in the case of delivery of reproductive material in the uterine cavity, the transfer catheter can be loaded with washed and prepared semen in the transfer tube and the transfer catheter can be placed in the inner catheter lumen.

The inner catheter can be extended and the everting balloon can evert and unroll through the cervix and into the uterine cavity. Concurrently or subsequently, the transfer catheter can be advanced through the inner catheter lumen into the uterine cavity. Once fully everted or when the transfer catheter becomes extended or exposed from the inner catheter and beyond the everting balloon membrane, the reproductive material in the transfer catheter can be deposited by a syringe, squeeze bulb, piston, or other pressure system. A second delivery catheter, such as a second insemination, IVF, or drug delivery catheter can be concurrently inserted into the inlet port or a second inlet port. The second delivery catheter can be deployed to the target site concurrent with or subsequent to the transfer catheter. The embryo transfer catheter can advance distally within the everting balloon and the inner catheter lumen. The transfer catheter can deposit the reproductive material (e.g., sperm) within the uterine cavity.

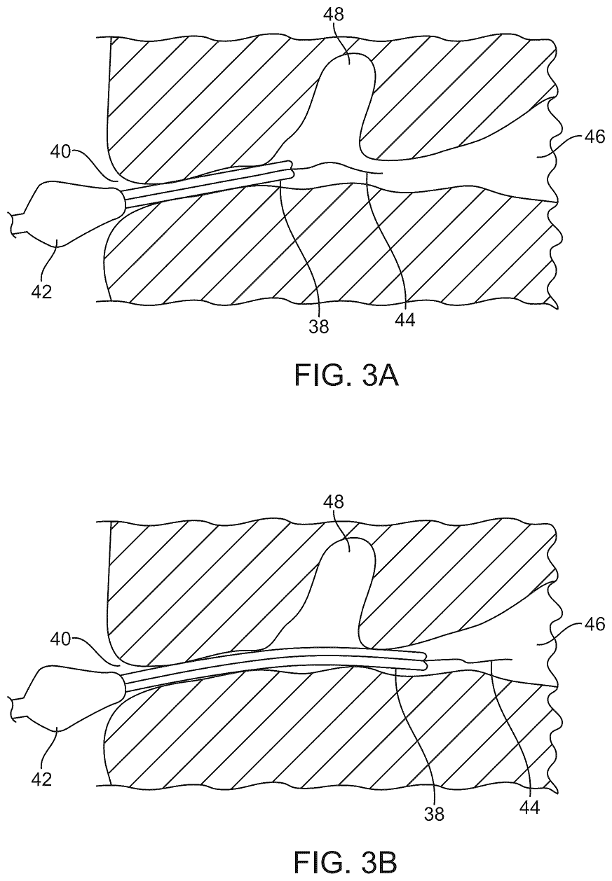

FIG. 3A illustrates a cross-sectional view of a flexible tip guidance wire extending beyond the distal terminal end of the everting balloon membrane during the eversion process directing the everting balloon system beyond a cul-de-sac in the endocervical canal or endocervix. (The everting balloon membrane distal end can be at or immediately adjacent to the cul-de-sac in the endocervical canal.) The flexible tip guidance wire distal end can be translatably advanced beyond the opening of the cul-de-sac and is positioned within the entrance or opening towards or within the uterine cavity. The delivery catheter system can be equipped with a flexible tip guidance wire that allows the physician to steer or direct the leading edge of the balloon to the correct path within the uterus, for example, to facilitate access within the uterine cavity and through the cervical canal. The delivery catheter system can be used, for example, when a defect, such as a C-section defect or scar, cul-de-sac, or crypt is present within the endocervix. Such defects can be visible via transabdominal or transvaginal ultrasound. The echogencitiy of the delivery catheter is enhanced by pressurization fluid, or air, or a combination of both that creates echogenic density differences that are visualized by ultrasound. The flexible tip guidance wire can be introduced beyond the cul-de-sac opening and towards the uterine cavity or target site, for example, to avoid the defect or cul-de-sac. The internal balloon pressure can be reduced or eliminated, for example, to advance the flexible tip guidance wire beyond the distal end of the everting balloon membrane. With everting balloon pressure low or at zero, the flexible tip guidance wire can be threaded through the deflated balloon membrane and advanced beyond the cul-de-sac opening. Once the flexible tip guidance wire is advanced beyond the opening and towards the target site, the everting balloon membrane pressure can be re-established and the advancement of the inner catheter can continue until the leading distal end of the everting balloon moves past or distal to the cul-de-sac opening.

FIG. 3B illustrates a cross-sectional view of a flexible tip guidance wire distal end distally beyond the everting balloon membrane distal end at the completion of the eversion process beyond a cul-de-sac in the endocervical canal. In this view, the everting balloon system has been advanced towards and within the uterine cavity without entering the cul-de-sac. Once past the opening and towards the uterine cavity or target location, the flexible tip guidance wire can be removed once full eversion is complete, or prior to that by reflating the everting balloon pressure to allow removal of the flexible tip guidance wire.

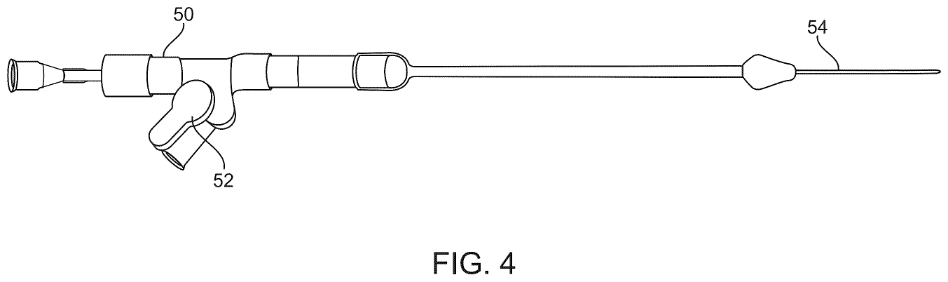

FIG. 4 illustrates a variation of the everting balloon system with a stopcock configuration for maintaining pressurization. The everting balloon membrane or system can be fully everted. The stopcock is placed on the Y-fitting connector which can be used by the physician to hold the everting balloon system during the procedure. The location of the stopcock can provide finger-tip control of the pressurization state of the everting balloon system. At the completion of the eversion step for the inner catheter, the pressurization state of the everting balloon membrane can be quickly removed. The removal of the pressurization state can occur prior to, during, or after the insertion of the embryo transfer catheter. Alternatively, the removal of the pressurization state can occur prior to, during, or after the deposition of the embryo(s) from the embryo transfer catheter. Yet further, the removal of the pressurization state can occur prior to, during, or after the removal of the embryo transfer catheter. Once the pressurization state is removed from the everting balloon system and after the embryo(s) have been deposited within the uterine cavity, the entire everting balloon system can be withdrawn from the uterine cavity.

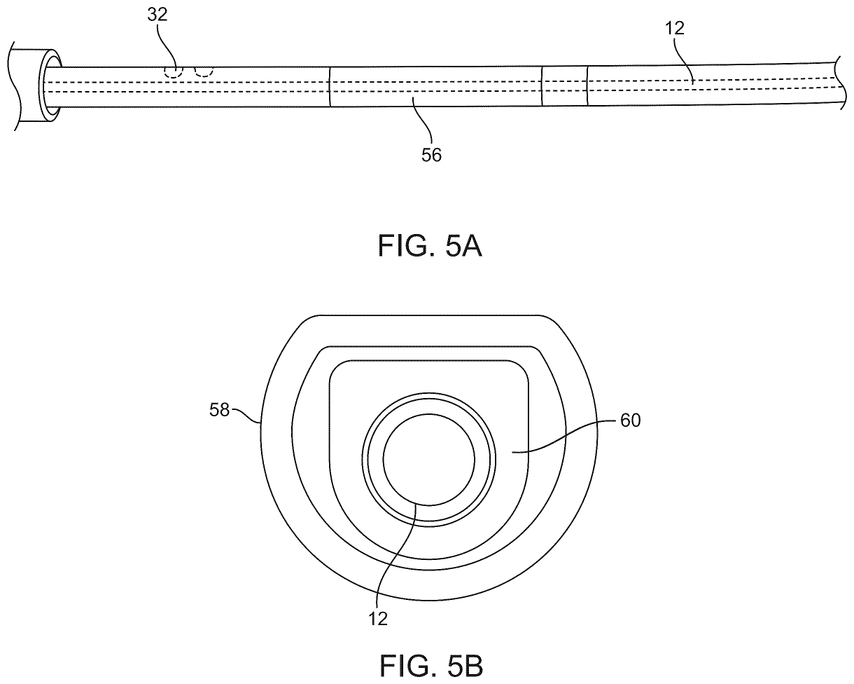

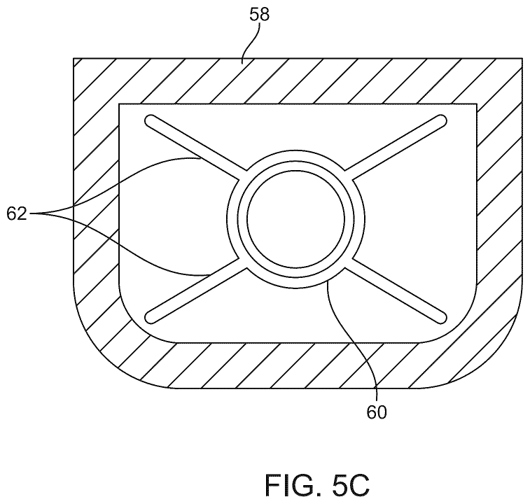

FIG. 5A illustrates a variation of the everting balloon system with an internal alignment mechanism that can prevent rotation and spinning of the balloon membrane, for example, with respect to the delivery catheter. The internal alignment mechanism can have or be an alignment piece. The internal alignment mechanism can restrict or eliminate the twisting of the balloon system about itself and/or with respect to the delivery catheter. Multiple twists within the balloon system can hinder the advancement of the embryo transfer catheter or other instruments and tools through the everting balloon system. The alignment piece can be located within the outer tubing distal to the Y-fitting and stasis valve that maintains pressurization within the everting balloon system while the inner catheter is being advanced or retracted during the eversion process.

FIG. 5B illustrates a cross-sectional axial view of the internal alignment mechanism and mating geometry of the delivery catheter tubing. The radially inner and/or outer surface of the delivery catheter outer tubing can be D-shaped. The alignment piece on the inner catheter can be keyed (e.g., having a somewhat similar D-shape to the outer tubing) within the D-shape of the outer tubing to restrict or eliminate the rotation of the inner catheter in relation to the outer tubing. The alignment piece can be made from a material with a lubricous coating, Teflon, or other material that reduces the friction of the alignment piece when being moved in the outer tubing.

The outer tubing outer and/or inner surface can have a D-shape, oval, elliptical shape, or combinations thereof, with a mating D-shape, oval, elliptical shape, or combinations thereof, on the alignment piece that can restrict or eliminates the rotation of the inner catheter in relation to the outer tubing.

The alignment piece shape can be configured as the external surface throughout the entire inner catheter tubing body. The shape of the external surface would in this configuration can mate with the internal geometry of the outer tubing. The surfaces can key into each other to restrict or eliminate the rotation of the inner catheter to the outer tubing and the stasis valve can conform or fit to the external surface of the inner catheter to maintain pressurization during the eversion process. As an example, the inner catheter tubing can be configured with a rail surface or protrusion that mates or keys with one or more receptacles within the outer tubing internal geometry.

FIG. 5C illustrates another embodiment of the alignment piece configured as a spline that mates within the internal geometry of the outer tubing. The spline outer surfaces engage the internal geometry of the outer tubing to restrict or eliminate the rotation of the inner catheter in relation to the outer tubing. The spline surfaces present minimal edges or corners that reduce the amount of surface area contacting the internal walls of the outer tubing. The reduction of surface area reduces the friction of the alignment piece when moved within the outer tubing.

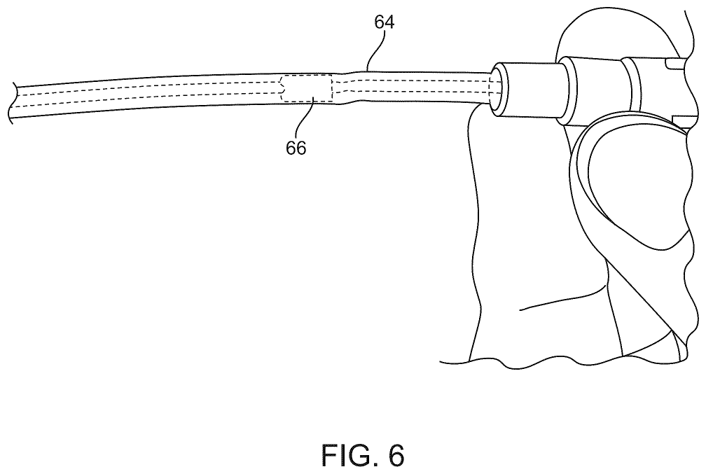

FIG. 6 illustrates a variation of an everting balloon system with an internal mechanism stopper that prevents over-elongation of the inner catheter during balloon inversion with a delivery catheter outer tubing crimp on the outer tubing of the delivery catheter. In use during the eversion and inversion procedure, or during the preparation of the everting balloon system, the end user or physician can inadvertently retract the everting balloon system and over-extend the balloon membrane. The over-extension can stretch, weaken, or damage the balloon membrane. Visual indicators or markings are useful but may not prevent over-extension if the end user is not diligent or is within a setting in which the indicia is readily visual. The stopper is located on the inner catheter tubing body and is positioned at point where full inversion has occurred. At full inversion, the stopper can contact a mechanical detent, crimp, stop, or the distal end of the Y-fitting connection, and prevents further retraction of the inner catheter thereby eliminating the over-extension of the balloon membrane beyond the full inversion state. The stopper could mechanically contact other mechanical structures built into the outer tubing such as a crimp as shown in FIG. 6, or a reduction in internal diameter of the outer tubing, in which the stopper would engage the crimp or reduction in internal diameter of the outer tubing to physically prevent further retraction of the inner catheter beyond the full inversion state.

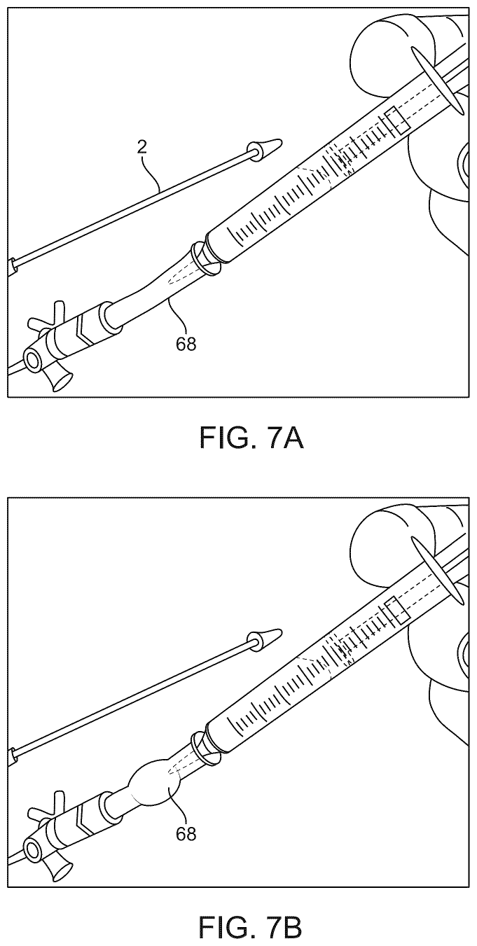

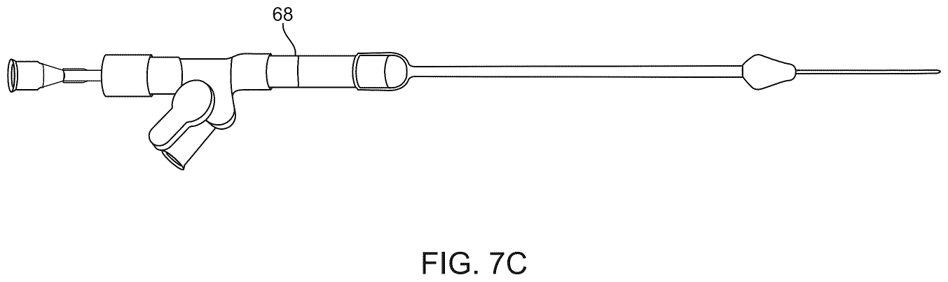

FIGS. 7A, 7B and 7C illustrate everting balloon systems with a compliant pressurization apparatus that can provide a pre-determined pressure within the catheter system with an indicator to the user that system is at the appropriate operating pressure.

FIG. 7A illustrates a compliant member built within the everting balloon system. The compliant member can be filled or instilled with a fluid from a syringe attached to the compliant member. The compliant member can be configured as a separate component or accessory to assist the end user in preparing the everting balloon system. As the pressurization of the everting balloon system occurs, compliant member inflates and becomes a visual indicator that the system contains pressure. The compliant member can be made from silicone tubing or balloon. Other elastomeric materials such as polyurethane, rubber, latex, configured as tubing or balloons are possible.

FIG. 7B illustrates that the compliant member can expand radially and lengthwise upon the influence of instilled fluid media under pressure. The expansion of the tubing walls of the compliant member can dampen the fluid pressure within the everting balloon system. This can allow for variances in the amount of fluid instilled by the end user that could impact the pressure rise in the everting balloon system. The amount of air in the everting balloon can provide some compliance to the everting balloon system and hence the compliant member can provide a range of fluid volumes without exceeding the recommended working pressure of the everting catheter system.

The everting catheter system can operates in a pressure range, for example, of about 2 to 4 atmospheres of pressure with a nominal pressure of about 3 atmospheres. For advancement within the cervical canal and into the uterine cavity, removing any residual air within the everting balloon system can be performed before, during, and/or after the eversion process. This can be used, for example, in situations with tight or stenotic cervices. To achieve a working pressure of 3 atmospheres, a pressure gauge and/or inflation device (e.g., with a pressure gauge) can be connected to the everting balloon system. To achieve a working pressure of 3 atmospheres, an exact fluid volume amount can be prescribed to the everting balloon system that can be instilled by the end user prior to end use. This can accomplish a working pressure of 3 atmospheres, for example, by measuring fluid volumes and the amount of air in the everting balloon system. The attachment of the compliant member to the everting balloon system can accomplish consistent fluid pressures within a wide range of fluid volumes, for example, providing a large tolerance to end user diligence during the catheter preparation process.

For example, a compliant member can be attached to an everting balloon system with a recommended fill volume of 3 cc of fluid. For test purposes while measuring preparing the everting catheter system with varying amounts of fluid volume, the internal pressure of the system does not alter (much) beyond the nominal pressure of 3 atmospheres and in all fluid volumes, even when the fluid volume is intentionally doubled beyond the instructed amount, the internal pressure of the everting balloon system remains within the operating working range of the system. For this test, the compliant member can be constructed with 50 durometer silicone tubing with a 0.250″ ID and a 0.500″ OD and a 1.5 cm length of silicone tubing. At the ends of the silicone tubing can be male and female luer connectors with attachment rings to mechanically adhere the silicone tubing to the luer connectors. In practice for this construction of the compliant member, as the silicone tubing is filled with fluid, the radial walls can expand and the overall length of the silicone tubing can increase in response to the increasing volume of fluid. Since the additional fluid volume can be accommodated by the compliant member, the internal fluid pressure of the everting balloon member can plateau at or near the desired nominal pressure amount.

| Fluid Volume Within | Resultant Internal |
|---|---|
| Everting Balloon | Pressure Within |
| System and | the Everting |
| Compliant Member | Balloon System |
| 3 cc of saline | 3.0 atmospheres |
| 4 cc of saline | 3.0 atmospheres |
| 5 cc of saline | 3.2 atmospheres |
| 6 cc of saline | 3.3 atmospheres |

As seen in the above data table, the resultant internal pressure can remain within the range of 2 to 4 atmospheres and at or near a nominal pressure of 3 atmospheres. In this set of experiments and with this configuration of compliant member, a fluid volume of 2X the amount yielded only a 10% increase in internal pressure. By altering the durometer, elastormeric properties, length and wall thickness of the compliant, other nominal pressure amounts can be obtained. The compliant member can provide a safety margin against over-pressurization that can either damage the balloon system, and/or provide an everting balloon system that operates outside of its operational working parameters. The compliant member can be used in combination with a pressure relief valve in everting balloon systems that have more critical or tight pressure tolerances, or where internal pressure changes due to operator or anatomical factors can create internal pressures that go beyond the desired performance specification.

FIG. 7C illustrates that the compliant member can be located on the outer tubing of the everting balloon system. The compliant member can be on the proximal end of the delivery catheter. The compliant member can inflate with internal pressure. The location of the compliant member can provide a visual indicator to the physician on the pressurization state of the everting balloon system and impacts the internal pressure of the everting balloon system due its compliance properties. The compliant member can be made from silicone, polyurethane, PVC, rubber, latex, or other elastomeric material and can be shaped as a tube or a preformed balloon. In this location, the entire compliant member can be held by the physician during use. In situations where a higher internal pressure is desired, the entire compliant member can be grasped and squeezed while maintaining positional control of the everting balloon system. Squeezing the compliant member circumferentially can create small rises in the internal pressure of the everting balloon system that may be advantageous for advancing the everting balloon through tight or narrow passages. In addition, relaxing the grasp of the compliant member would instantly return the compliant member and the everting balloon system to the pervious operating pressure range. In some applications, the ability to pulse the compliant member and thereby provide pulsatile pressure spikes within the everting balloon system may be advantageous for tight or narrow passageways in which advancement of the everting balloon is desired in small and discrete steps, or with minor increases in internal pressure.

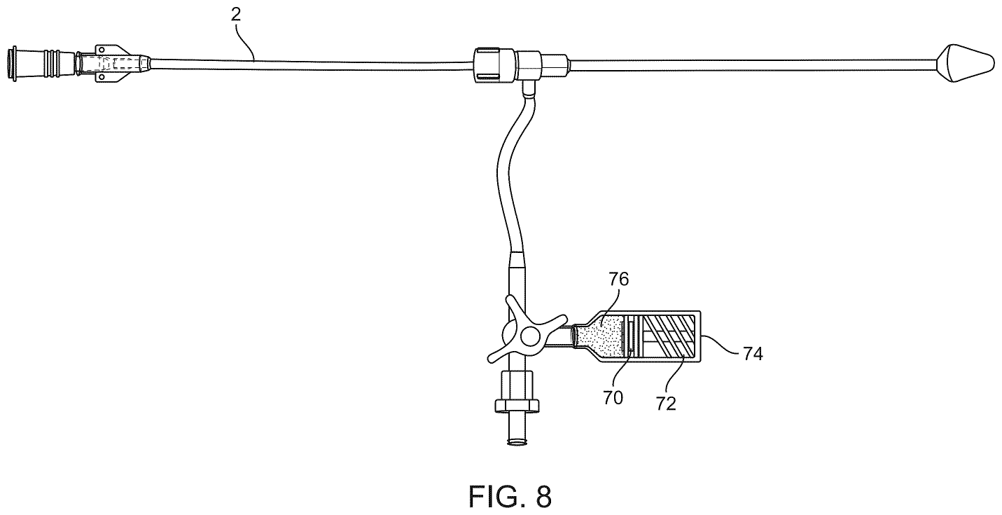

FIG. 8 illustrates that the everting balloon system can have a mechanism that can automatically pressurize the everting balloon system to a predetermined amount. In a side view, a fluid cartridge attached to and in controllable (e.g., via a luer lock or valve) fluid communication with the everting balloon system can have a syringe plunger and spring assembly. The fluid cartridge can have a chamber that can be filled with a fluid that can be used to supply internal pressure to the everting balloon. The syringe plunger and spring assembly can have a spring that can drive the plunger into the chamber with a known spring constant or K factor. The spring and k factor can be selected and configured to deliver a predetermined internal pressure to the everting balloon system. The spring can provide compliance to the everting balloon system to maintain the internal pressure within the operating range and the spring, like the compliant member, can be responsive to changes in fluid volume, the everting balloon system itself as it everts and inverts, and any anatomical forces acting on the everting balloon system.

The syringe plunger and spring assembly can be substituted for or used in combination with a syringe plunger and air pressure canister in which the air canister with a predetermined internal gas pressure replaces the spring. Pressure from the air canister can act on the plunger and drive the fluid volume within the everting balloon system to a predetermined internal pressure range. Air pressure canister can be prefilled with CO2 gas, air, other inert gas, or combinations thereof.

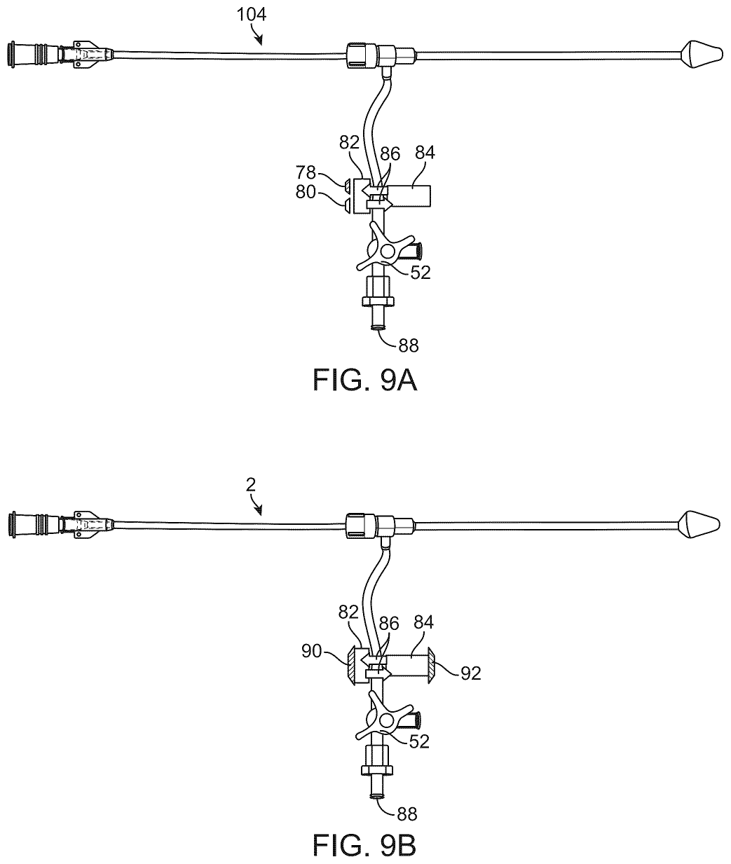

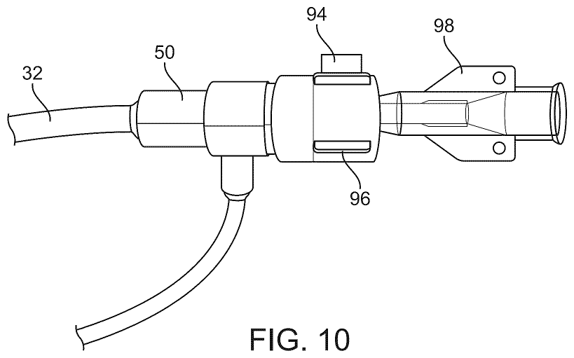

FIG. 9A illustrates an everting balloon system with an integral pressurization system that can provide an indicator and the ability to quickly shift the pressurization state of the balloon system from pressurized to non-pressurized at the fully everted state of the everting balloon system via actuating trumpet valves for fluid from constant pressure source to a separate fluid reservoir. Actuation of trumpet valves can direct fluid back into constant pressure source through one-way valves. For example, a first trumpet valve can release into the fluid reservoir, and a second trumpet valve can return to a constant pressure source. A stopcock is used to prepare and fill the everting balloon system with fluid. The system can also have one-way valves. The one-way valves can be within the trumpet valves (or the trumpet valves themselves) or can be separate from the trumpet valves. The everting balloon system can have a fill port.

FIG. 9B illustrates an everting balloon system with an integral pressurization system that can provide an indicator and the ability to quickly shift the pressurization state of the balloon system from high pressurization to low pressurization, and back to high pressurization, or multiple intermediate states of pressurization, during the eversion process. A switch valve diverter and diaphragm on the fluid reservoir can open and close the fluid pathway into the everting balloon system from the constant pressure source and fluid reservoir. Depressing the plunger or diaphragm of the fluid reservoir returns the fluid volume back into the everting balloon system and in communication with the constant pressure source. While fluid is diverted into the fluid reservoir, the internal pressure within the everting balloon system drops to, or at, nears zero atmospheres. Depressing the diaphragm plunger of the fluid reservoir can push fluid through a one-way valve into the constant pressure source chamber. Manual depression forces on the diaphragm can be facilitated by the flexure of the diaphragm surface from a convex profile to a concave profile as the fluid is pushed through the one-way valve and into the constant pressure force chamber. Fluid going into the constant pressure force chamber can flow through a one-way valve to enter the chamber. Once the diverter is flipped back to the everting balloon system, the constant pressure source can instill the fluid back into the everting balloon. Other combinations of one-way valves, check valves, or turn valves are possible to allow fluid pressure in the everting balloon systems to change from an operating pressure state or a zero pressure state quickly without having to reconnect to the everting catheter to a separately supplied fluid source, or without moving the position of the everting catheter within the bodily cavity.

The constant pressure source could be configured to supply varying amounts of force for providing the internal pressure of the everting catheter system. The constant pressure source can be supplied with a constant pressure regulator that can modulate the amount of internal pressure being supplied to the everting balloon system. Pressure modulation can provide change from 3 atmospheres of pressure to 2, 1, or 0.5 atmospheres of pressure which can still provide the everting balloon with structural shape but reduces the amount of eversion force, or the overall diameter of the everting balloon. For example, the everting balloon can have its internal pressure modulated from 3 atmospheres of pressure at a point of nearly complete eversion but would then have the internal pressure modulated to 0.5 or 1 atmospheres of pressure as the embryo transfer catheter is being loaded by the embryologist, or when the embryo transfer catheter is being traversed through the inner catheter, or at as the entire everting catheter is inverted or removed from the uterine cavity without inverting the balloon back into the delivery catheter. Other degrees of pressure are possible with fingertip control of the physician without having to use an inflation device hooked up to the everting catheter system.

FIG. 10 illustrates an everting balloon system with a mechanism that stabilizes the inner catheter at the full eversion stage and provides an indicator to the user that catheter system is at the appropriate step in the process for embryo transfer. The inner catheter and the everting balloon can reach full eversion when the inner catheter proximal hub contacts the cap of the Y-fitting of the delivery catheter. Receptacle on Y-fitting cap is configured to accept and mate with the distal surface of the proximal hub. Contact of the inner catheter proximal hub to the cap of the Y-fitting elevates a pop up locking tab as a visual indicator of the engaged position. (The pop up locking tab is shown in the elevated and engaged position in FIG. 10.) Mating action can be an audible or palpable, or both, as the two surfaces engage and lock. For the embryo transfer procedure, when the two surfaces engage and lock, the embryologist would provide the embryo transfer catheter for traversing through the inner catheter. Depression of the pop up locking tab to unlock would free the inner catheter proximal hub from the mating surface. The two surfaces can also engage without locking, or engage with a mechanical or friction fit that can be overcome by slight retraction by the physician. As another embodiment, the mating action of the two surfaces could also mechanically open turn valve on the Y-fitting to remove internal pressure within the everting balloon system to reduce profile of the everting balloon once the full eversion process is completed.

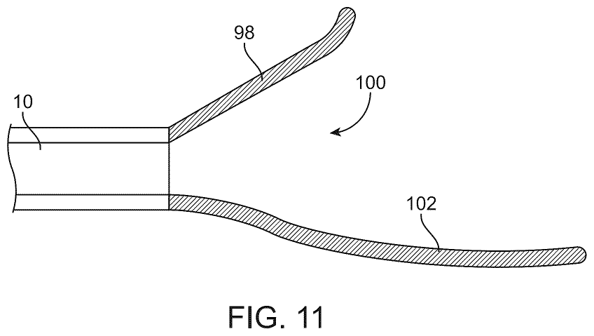

FIG. 11 illustrates in a side cross-sectional view an everting balloon system with a proximal hub connector that aids physician and embryologist in delivering the embryo transfer catheter to the delivery catheter. The inner catheter proximal hub can have a large funnel opening to provide an easier target for the embryologist or the physician to place the distal end of the embryo transfer catheter into the everting catheter system. Funnel of the proximal hub can also have a posterior extension that provides a platform for resting the proximal end of the embryo transfer catheter during the final steps of embryo transfer catheter insertion, such as while inserting into the everting catheter. This may be particularly beneficial with embryology syringes that are heavy in weight, such as glass syringes, that could create extra downward forces on the embryo transfer catheter.

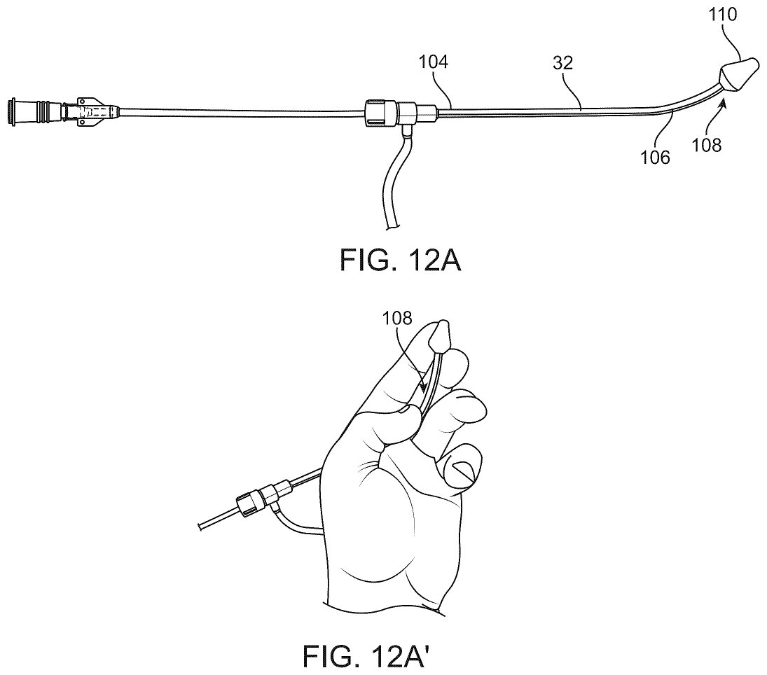

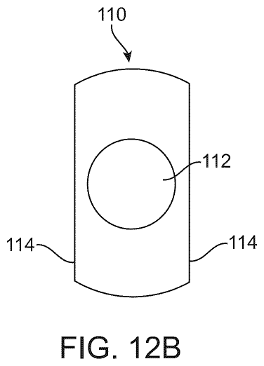

FIGS. 12A and 12A′ illustrate an everting balloon system shaped with distal end features that facilitate uterine access without the need for a speculum. During embryo transfer procedures, minimizing the manipulations to the patient's uterus, cervix, and vagina is both more comfortable to the patient but can also have a significant role in reducing the amount of uterine contractions that could spontaneously arise during a procedure as a result or response to the manipulations. Uterine contractions can have a deleterious effect to the implantation of embryos during a procedure. The insertion of a speculum itself has been demonstrated to elicit uterine contractions and is uncomfortable to the patient. The embodiment of the everting catheter system in FIGS. 12A and 12B also facilitates the use of a transvaginal ultrasound probe during the embryo transfer procedure. Transvaginal ultrasound provides greater vision quality than an abdominal ultrasound in which the abdominal ultrasound probe needs to provide sound waves through the pelvic region of the patient which may have varying degrees of abdominal fat. Also abdominal ultrasound is enhanced by the patient having a full bladder which can also add to the discomfort to the procedure. The use of a transvaginal ultrasound probe during an embryo transfer procedure is difficult since existing embryo transfer catheter systems require a speculum for insertion of the device into the cervix. The embodiment illustrated in FIGS. 12A and 12B is designed to provide rigidity to enter into the vagina and press off the posterior surface of the vagina. Angulation or curvature of the delivery catheter distal end can direct the distal tip of everting catheter towards to cervical os. As illustrated in FIG. 12B, acorn tip is shaped for placement alongside the transvaginal ultrasound probe by having flat surfaces on either side of the acorn tip. In practice the physician would place the transvaginal probe into the vagina and alongside the cervix. The everting catheter system would be introduced alongside the transvaginal probe until the acorn tip is at the exocervix. The presence of the transvaginal ultrasound probe would create access, and in most cases, room in the vagina for visual confirmation of placement at the exocervix without the need for a speculum. The everting balloon would be then placed into the endocervical canal. For an everting catheter system, the portion of the everting catheter that contacts the endocervix and uterine cavity is all contained within the delivery catheter and does not contact the surfaces or fluids in the vagina, thus further obviating the need for a speculum during the procedure. Referring back to FIG. 12A, posterior side of delivery catheter has a curved flexure support. Flexure support is designed to maintain distal end curvature for entry into the vagina and placement of the distal acorn tip at the exocervix. Flexure support has rigidity to push slightly downward in the vagina to retract vaginal tissues away from the cervix. Photo insert in FIG. 12A shows the curvature of the delivery catheter.

FIG. 12B illustrates a distal end with flat surfaces on both sides of the acorn tip facilitate placement along either side of the transvaginal ultrasound probe regardless of the physician being right or left handed. FIG. 12B illustrates the acorn tip can have a distal end hole and flat sides.

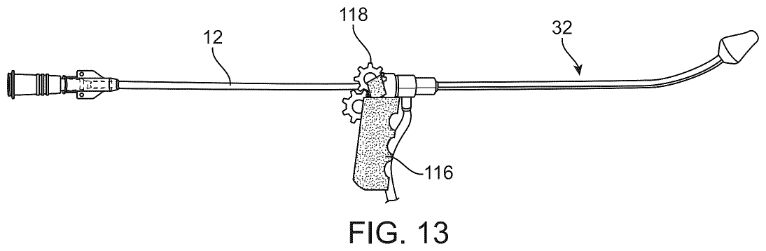

FIG. 13 illustrates in a side view of an everting balloon system with a handle and controller mechanism that controls the translation of the inner catheter. The handle can minimize the amount of overall working length of the everting catheter system without adding length to the everting catheter system. As an example, working space needed to place a handle within an everting catheter system can increase the overall length to the delivery catheter, inner catheter, and the embryo transfer catheter that needs to be placed within the system. Adding length to these systems can create handling issues within the embryology laboratory, for example, in labs in which the loading of embryos within the embryo transfer catheter is performed within a small incubator with side walls that will encroach on the handling of the embryology syringe and placement of the distal end of the embryo transfer catheter within the embryo dish within the incubator. The handle can reduce the amount of working length occupied by the handle and controller mechanism while still providing a one-handed operation to the advancement of the inner catheter during use. The controller mechanism can translatably advance and/or retract the inner catheter. The handle can contain gear wheel controller mechanism for engaging and translating the inner catheter during use. The handle has a posterior section that fits the palm and fingers of the physician without requiring the inner catheter to be placed through the handle portion for engagement with the controller mechanism. The handle can be a pistol grip with gear wheels actuated by the thumb. The handle can be incorporated into an everting catheter system for use with transvaginal ultrasound.

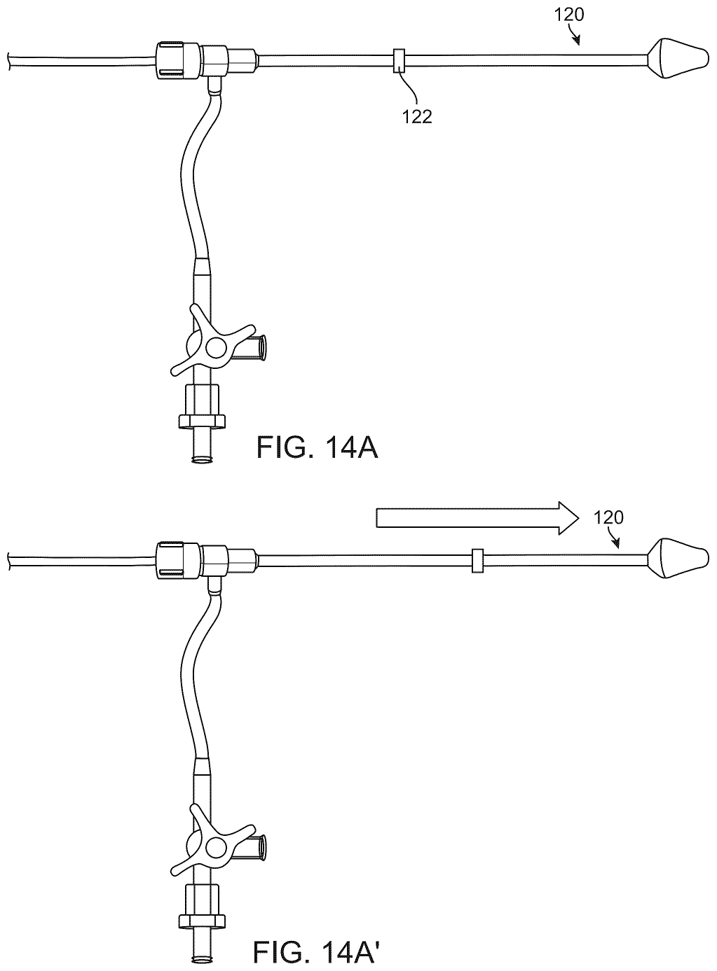

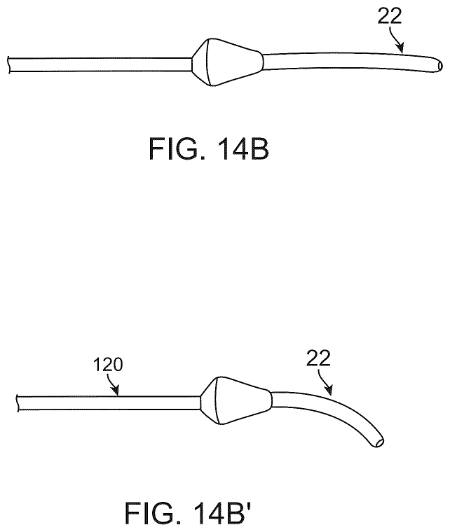

FIGS. 14A and 14A′ illustrate an everting balloon system with a translatable and adjustable distal end tip that can alter the working length of the everting balloon. The translatable and adjustable distal end tip can have a connector on its proximal end and an acorn tip on its distal end that can be advanced or retracted on the distal end of the delivery catheter. Advancement of the translatable and adjustable distal end tip, as shown in FIG. 14A′, can reduce the overall length of the everting balloon within the bodily cavity without impacting the markings on the proximal end of the embryo transfer catheter.

FIG. 14B illustrates the translatable and adjustable distal end tip at an extended position with resultant working length of the everting balloon and everting balloon membrane. As an example, an everting balloon system with a 5 cm long everting balloon has the translatable and adjustable distal end tip advanced 3 cm on the delivery catheter. As shown in FIGS. 14B′, the resultant new working length of the everting balloon in the body cavity is 2 cm. The connector on the proximal end of the translatable and adjustable distal end tip can be rotated to engage edges of D-shape tubing of the delivery catheter. Once rotated in the locked position, the translatable and adjustable distal end tip can be configured to no longer move or slide on the delivery catheter. Unlocking the connector by rotation can return movement to the translatable and adjustable distal end tip. Other types of connectors can include twist valves that resist movement by friction on the outer tube of the delivery catheter and can be untwisted to allow movement. Another example of a connector is a clip that has an engaged and disengaged position which is actuated by the user.

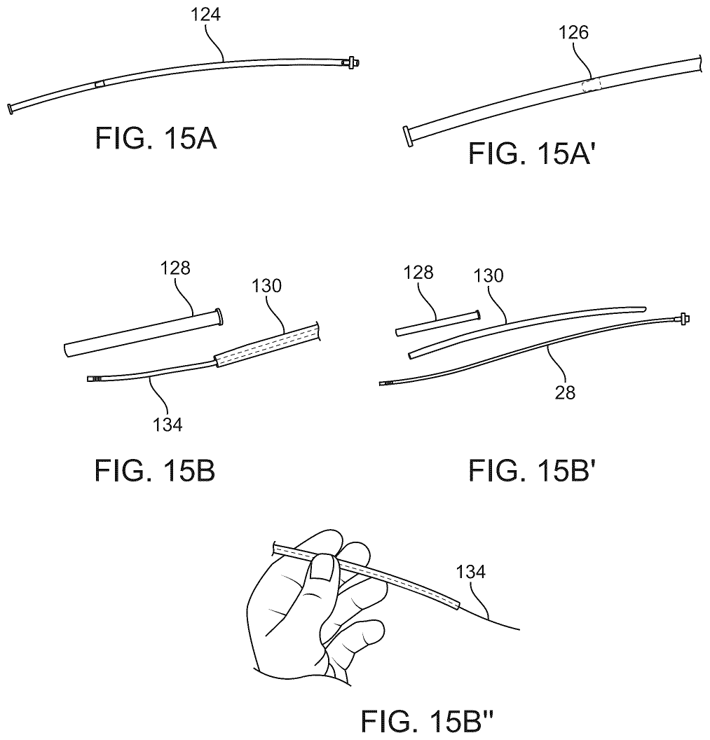

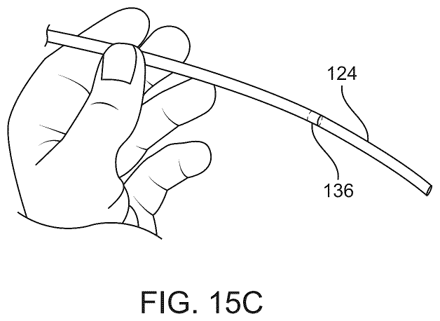

FIGS. 15A and 15A′ illustrate a protective tube system for the embryo transfer catheter that can facilitate handling and transport of the catheter. The protective tube can be shipped to the user assembled with two tube components with male and female connections attached to each other with the embryo transfer catheter within the lumen of the protective tube.

FIGS. 15B, 15B′ and 15B″ illustrate a protective tube system for the embryo transfer catheter in the detached configuration for the loading of embryos. The female connection of the tube component at a female end is separated from the male connection at a male end at a point near the embryo transfer catheter distal end, leaving a distal length of the embryo transfer catheter exposed for working under microscopic vision and the loading of embryo(s) and/or reproductive materials. FIG. 15B″ shows the handling of the embryo transfer catheter when loading embryos with an embryology syringe.

FIG. 15C illustrates a protective tube system for the embryo transfer catheter in the re-attached mode for the transport of the embryo transfer catheter. Once loaded with embryo(s) and reproductive materials, the female connection can be reattached to the male connection for transport to the patient and the delivery catheter system. The distal end of the embryo transfer catheter, for example with the reproductive material, can be radially covered by the protective tube system when the female end is attached to the male end. The reattachment connection point in the protective tube between the male and female ends can be separated, for example, to provide a mechanism to gain access to the distal end of the embryo transfer catheter for manipulation under a microscope or within an embryology incubator, and further be reattached for transport of the reproductive material to the patient and the completion of the embryo transfer procedure.

U.S. Pat. Nos. 9,028,401, issued May 12, 2015; 9,101,391, issued Aug. 11, 2015, 9,949,756, issued Apr. 24, 2018; U.S. patent application Nos. 14,495,726, filed Sep. 24, 2014; 14,525,043, filed Oct. 27, 2014; and U.S. Provisional Application Nos. 61/902,742, filed Nov. 11, 2013; 61/977,478, filed Apr. 9, 2014; 62/005,355, filed May 30, 2014; and 62/007,339, filed Jun. 3, 2014, are incorporated by reference herein in their entireties. The elements of the aforementioned patents and patent applications can be combined with those disclosed elsewhere herein.

Any elements described herein as singular can be pluralized (i.e., anything described as “one” can be more than one). Any species element of a genus element can have the characteristics or elements of any other species element of that genus. The media delivered herein can be any of the fluids (e.g., liquid, gas, or combinations thereof) described herein. The patents and patent applications cited herein are all incorporated by reference herein in their entireties. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

We claim:

1. A method for delivering matter into a uterine cavity comprising:
positioning an everting balloon system adjacent to a cervical canal, wherein the everting balloon system comprises a first catheter and an everting balloon attached to the first catheter, and wherein the first catheter has a catheter lumen and a distal port at a distal end of the catheter lumen, and a delivery catheter attached to an opposite end of the everting balloon from the first catheter, wherein the delivery catheter has a delivery catheter lumen;
everting the everting balloon in the cervical canal, wherein the everting comprises pulling the first catheter distally through the cervical canal, wherein the first catheter has a fixed alignment with the delivery catheter and everting balloon, wherein the fixed alignment is facilitated by an alignment piece within the delivery catheter lumen, wherein a cross-sectional geometry of the alignment piece is asymmetrical and configured to restrict rotation of the first catheter in relation to the delivery catheter, and wherein the everting comprises inflating the balloon distal to the first catheter;
pressurizing the everting balloon system with a compliant member to facilitate keeping an internal pressure within an operating range of 2 to 4 atmospheres, wherein the compliant member has a first longitudinal end and a second longitudinal end, wherein the compliant member comprises a 50 durometer silicone tubing, wherein the first and second longitudinal ends of the compliant member are open, and wherein the compliant member holds pressure such that upon everting the balloon, the compliant member maintains the pressure within the balloon; and
advancing a tool through the first catheter, wherein the tool comprises a therapeutic agent.

2. The method of claim 1, wherein the fixed alignment prevents twisting of the everting balloon during eversion and inversion of the everting balloon.

3. The method of claim 2, wherein the fixed alignment prevents over extension of the everting balloon during the inversion process.

4. The method of claim 1, wherein the delivery catheter has an inner surface, wherein a length of a shape of the inner surface of the delivery catheter matches a length of a shape of the alignment piece.

5. The method of claim 1, further comprising filling the compliant member with fluid from a syringe.

6. A method for delivering matter into a uterine cavity comprising:
everting a balloon in a cervical canal, wherein the balloon is attached to a first catheter, and wherein everting comprises pulling the first catheter distally through the cervical canal, wherein a proximal portion of the balloon has a proximal portion diameter between 2 mm to 4 mm for a length of between 5 mm to 10 mm from a distal terminal end of the first catheter, wherein a remainder of the balloon has a distal portion diameter within 4 mm to 7 mm;
pressurizing a balloon system with a compliant member to facilitate keeping an internal pressure within an operating range of 2 to 4 atmospheres, wherein the compliant member has a first longitudinal end and a second longitudinal end, wherein the first and second longitudinal ends of the compliant member are open, and wherein the compliant member holds pressure such that when the balloon everts, the compliant member maintains the pressure within the balloon; and
advancing a tool through the first catheter, wherein the tool comprises a therapeutic agent.

7. The method of claim 6, further comprising filling the compliant member with fluid from a syringe.

8. The method of claim 6, wherein the compliant member is made from silicone tubing.

9. A system for delivering matter into a reproductive tract of a female comprising:
a first catheter having a lumen and a distal lumen port, wherein the first catheter has a retracted configuration and an extended configuration;
an everting balloon attached to the first catheter, wherein at least a length of the everting balloon extends past a distal end of the first catheter when the first catheter is in the extended configuration;
a compliant member to facilitate keeping internal pressure within an operating range of 2 to 4 atmospheres, wherein the compliant member has a first longitudinal end and a second longitudinal end, wherein the first and second longitudinal ends of the compliant member are open, and wherein the compliant member holds pressure such that when the balloon everts, the compliant member maintains the pressure within the balloon;
a second catheter slidably located in the first catheter;
a stopper located around the second catheter and configured to prevent over extension of the everting balloon during inversion of the everting balloon;
a tool advanceable through the second catheter, wherein the tool comprises a therapeutic agent; and
wherein the second catheter comprises an alignment piece to prevent rotation of the second catheter in relation to the first catheter, wherein fluid can flow between the alignment piece and the first catheter, and wherein a cross-sectional geometry of the alignment piece is asymmetrical and is configured to restrict rotation of the first catheter in relation to the second catheter.

10. The system of claim 9, wherein the compliant member is made from silicone tubing.

11. The system of claim 9, wherein the compliant member is in fluid communication with a syringe.

12. The system of claim 9, wherein the first catheter has an inner surface, wherein a length of a shape of the inner surface of the first catheter matches a length of a shape of the alignment piece.

13. A matter delivery system for delivering matter into a reproductive tract of a female comprising:
a first catheter having a lumen and a distal lumen port, wherein the first catheter has a retracted configuration and an extended configuration; an everting balloon attached to the first catheter, wherein at least a length of the everting balloon extends past a distal end of the first catheter when the first catheter is in the extended configuration when the everting balloon is pressurized and extended to traverse the reproductive tract, wherein a proximal portion of the balloon has a proximal portion diameter between 2 mm to 4 mm for a length of between 5 mm to 10 mm from a distal terminal end of the first catheter, wherein a remainder of the balloon has a distal portion diameter within 4 mm to 7 mm;
a second catheter slidably located in the first catheter;
a stopper located around the second catheter and configured to prevent over extension of the everting balloon during inversion of the everting balloon;
a tool advanceable through the first catheter, wherein the tool comprises a therapeutic agent; and
an everting balloon system with a compliant member to facilitate keeping internal pressure within an operating range of 2 to 4 atmospheres, wherein the compliant member has a first longitudinal end and a second longitudinal end, wherein the first and second longitudinal ends of the compliant member are open, and wherein the compliant member holds pressure such that when the balloon everts, the compliant member maintains the pressure within the balloon.

14. The system in claim 13, wherein the compliant member is constructed from a resilient tubing material within the operating range of the matter delivery system.

15. The system of claim 14, wherein the compliant member is made from silicone tubing.

16. The system of claim 13, wherein the compliant member is placed onto the first catheter and provides a visual indication of the internal pressure of the matter delivery system.

17. The system of claim 16, wherein the compliant member is configured to provide additional pressure to the everting balloon by manually depressing or squeezing the compliant member during operation.

18. The system of claim 13, wherein the compliant member is in fluid communication with a syringe.
